(19) United States
(12) Patent Application Publication
(10) Pub. No.: US 2022/0284603 A1
(43) Pub. Date: Sep. 8, 2022

(54) System and Method for Producing an Image-based Registration of Surgically Excised Tissue Specimens

(71) Applicant: CytoVeris Inc., Farmington, CT (US)

(72) Inventors: David Fournier, Northborough, MA (US); Kevin Didden, Wallingford, CT (US); Alan Kersey, South Glastonbury, CT (US)

(21) Appl. No.: 17/687,283

(22) Filed: Mar. 4, 2022

Related U.S. Application Data
(60) Provisional application No. 63/156,486, filed on Mar. 4, 2021.

(51) Int. Cl. G06T 7/30; G06T 19/00; G16H 10/40

(52) U.S. Cl. CPC G06T 7/30; G06T 19/006; G16H 10/40; G06T 2207/30096; G06T 2207/10012; G06T 2207/10028; G06T 2210/41

(57) ABSTRACT

A system and method of producing an image-based registration of an excised tissue specimen for histopathological examination is provided. The method includes: a) producing a three-dimensional image of an excised tissue specimen having a plurality of surfaces at a first location, the excised tissue specimen having an in-vivo orientation prior to being excised; b) producing orientation information relating to the orientation of the plurality of surfaces of the excised tissue specimen relative to the in-vivo orientation of the tissue specimen prior to being excised; c) wherein the excised tissue specimen is transported to a second location and is positioned; and d) using the three-dimensional image and the orientation information to confirm the excised tissue specimen positioned at the second location is in the in-vivo orientation.

This application claims priority to U.S. Patent Appln. No. 63/156,486 filed Mar. 4, 2021, which is hereby incorporated by reference in its entirety.

### BACKGROUND OF THE INVENTION

### 1. Technical Area

The present disclosure relates to systems and methods for processing excised tissue specimens for pathology in general, and to systems and methods for such processing that utilize digital images to provide information relating to the orientation of the excised tissue specimen as removed from a subject in particular.

### 2. Background Information

For many decades, the reference method for the diagnosis of cancer has been histopathological examination of tissues using conventional microscopy. This process is known as Surgical Pathology. In Surgical Pathology, tissue specimens can be produced from surgical procedures (tumor resection), diagnostic biopsies or autopsies. Once removed from the subject, the tissue specimens are typically subjected to a process that includes dissection, fixation, and cutting of the tissue specimen into precisely thin slices which are stained for contrast and mounted onto glass slides. The slide mounted tissue specimen is subsequently examined by a pathologist under a microscope, and the pathologist's interpretation of the tissue becomes the basis for the pathology “read” of the tissue specimen.

Once a tissue specimen is removed from a subject, it is often the case that the specimen is handled by multiple different parties with each party being a link in the “chain of custody” of the specimen. For example, the specimen may pass from a surgeon to a grossing lab technician, and then to a histotechnologist, and then finally to a pathologist. Each of these entities provides a different function; e.g., the grossing lab technician may be tasked with “macro” tasks (hence the name “grossing”) such as dimensionally measuring the specimen, weighing the specimen and providing a description of the specimen (e.g., shape, color, texture, etc.), as well as applying colorant to the surfaces of the specimen to indicate surface orientation. The histotechnologist may be tasked with preparing and cutting the specimen into thin sections (e.g., using a microtome) and then mounting the tissue slices onto a slide. The pathologist performs the pathology “read” of the slide mounted tissue and prepares a report for the surgeon. Of course, the above described “chain of custody” and its participants are provided here as a non-limiting example of a process that may be used to illustrate that more than one party is typically involved and each represents the potential for a miscommunication of information. For example, when a specimen is excised it is common practice for a surgeon to apply one or more sutures to the excised specimen each operating as a “marker”. Notes and diagrams produced from the surgeon that accompany the specimen to the grossing lab may indicate the orientation of the specimen based on the position of the sutures; i.e., designating the medial, lateral, posterior, anterior, superior, and inferior surfaces. Based on the notes and “markers”, the grossing lab technician may then apply a different colorant to each respective surface. The diagram shown in FIG. 1 illustrates an example of how surface orientations may be indicated by different colors. The distinguished specimen surfaces (“margins”) may be then used by the histotechnologist to orient the tissue slices and by the pathologist to understand the relative positions of the slices from the specimen.

Ultimately, it is almost always useful and can be quite important to know the orientation of the tissue specimen as excised from the subject. Knowing the correct orientation of the tissue specimen provides information regarding the margins of the specimen and the cavity from which the specimen was excised; e.g., a malignant lesion may extend across a boundary of surgical excision. If subsequent pathological analysis determines malignant tissue on the surface of a specimen, knowing the corresponding in-vivo surface will be important.

The conventional pathology process described above and its transfer of information from party to party in the chain of custody may, on occasion, result in a miscommunication or misinterpretation. For example, a miscommunication or misinterpretation may result in an error regarding the actual orientation of the excised specimen relative to the indicated orientation of the tissue specimen read by the pathologist. What is needed is an improved method for tracking the excised specimen through the pathology process and/or one that facilitates the pathology process and record keeping of the pathology process.

### SUMMARY OF THE INVENTION

According to an aspect of the present disclosure, a method of producing an image-based registration of an excised tissue specimen for histopathological examination is provided. The method includes: a) producing a three-dimensional image of an excised tissue specimen having a plurality of surfaces at a first location, the excised tissue specimen having an in-vivo orientation prior to being excised; b) producing orientation information relating to the orientation of the plurality of surfaces of the excised tissue specimen relative to the in-vivo orientation of the tissue specimen prior to being excised; c) wherein the excised tissue specimen is transported to a second location and is positioned; and d) using the three-dimensional image and the orientation information to confirm the excised tissue specimen positioned at the second location is in the in-vivo orientation.

In any of the aspects or embodiments described above and herein, the second location is remote from the first location, and the method may further include communicating the three-dimensional image and the orientation information to the second location for use in the confirmation step.

In any of the aspects or embodiments described above and herein, wherein the three-dimensional image may be displayed on a user portal at the second location.

In any of the aspects or embodiments described above and herein, wherein the method may further include producing a second three-dimensional image of the excised tissue specimen at the second location, and using the second three-dimensional image in the confirmation step.

In any of the aspects or embodiments described above and herein, wherein the method may further include imaging the excised tissue specimen at the second location and overlaying the three-dimensional image of the excised tissue specimen onto the excised tissue specimen being imaged at the second location.

In any of the aspects or embodiments described above and herein, wherein the process of overlaying the three-dimensional image of the excised tissue specimen onto the excised tissue specimen being imaged at the second location may be performed using augmented reality.

In any of the aspects or embodiments described above and herein, wherein the process of overlaying the three-dimensional image of the excised tissue specimen onto the excised tissue specimen being imaged at the second location may include alignment of a plurality of markers.

In any of the aspects or embodiments described above and herein, wherein the excised tissue specimen is sliced into a plurality of sections at the second location, and the method may further include producing a three-dimensional image of each of the plurality of sliced sections.

In any of the aspects or embodiments described above and herein, wherein the step of producing a three-dimensional image of an excised tissue specimen may utilize a stereo digital camera.

In any of the aspects or embodiments described above and herein, wherein the step of producing a three-dimensional image of an excised tissue specimen may utilize a scanning digital camera, or a LIDAR camera, or an imaging system that utilizes time-of-flight technology.

In any of the aspects or embodiments described above and herein, wherein the step of producing a three-dimensional image of an excised tissue specimen may include imaging the excised tissue specimen to produce a plurality of different perspective images and forming the three-dimensional image using the plurality of different perspective images.

In any of the aspects or embodiments described above and herein, wherein the method may further include virtually adding a plurality of different colorants to the plurality of surfaces within the three-dimensional image of an excised tissue specimen, wherein each said different colorant is added to a respective one of the plurality of surfaces.

According to another aspect of the present disclosure, a system for image-based registration of an excised tissue specimen for histopathological examination is provided. The excised tissue specimen has a plurality of surfaces and an in-vivo orientation prior to being excised. The system includes an imaging system, a first user portal, and a second user portal. The imaging system is configured to selectively produce a three-dimensional image of an excised tissue specimen. The first user portal is located in a first location. The second user portal is located in a second location remote from the first location. The first user portal is in communication with the imaging system and the second user portal. The first user portal includes an input device configured to permit entry of orientation information relating to the orientation of the plurality of surfaces of the excised tissue specimen, and the first user portal is configured to communicate the three-dimensional image and the orientation information to the second user portal. The second user portal is configured to permit a user to use the three-dimensional image and the orientation information to confirm the excised tissue specimen is in the in-vivo orientation.

In any of the aspects or embodiments described above and herein, the second user portal may include a display configured to selectively display the three-dimensional image.

In any of the aspects or embodiments described above and herein, the system may further include a second imaging system at the second location. The second imaging system is in communication with the second user portal. The second imaging system may be configured to produce a second three-dimensional image of the excised tissue specimen in a format that permits comparison between the first three-dimensional image and the second three-dimensional image.

In any of the aspects or embodiments described above and herein, the system may further include a second imaging system at the second location. The second imaging system is in communication with the second user portal. The second imaging system may be configured to image the excised tissue specimen and overlay the three-dimensional image onto the imaged excised tissue specimen.

In any of the aspects or embodiments described above and herein, the system may further include a second imaging system at the second location. The second imaging system is in communication with the second user portal. The second imaging system may be configured to overlay the three-dimensional image onto the imaged excised tissue specimen using augmented reality.

In any of the aspects or embodiments described above and herein, the imaging system may include a digital camera, a stereo digital camera, a scanning digital camera, a LIDAR camera, or an imaging system that utilizes time-of-flight technology, or any combination thereof.

In any of the aspects or embodiments described above and herein, the imaging system may include a digital camera and the second user portal may be configured to form the three-dimensional image using images from the digital camera.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagram (sometimes referred to as an “inking chart”) illustrating the orientation of specimen surfaces and the colors used to indicate the respective surfaces.

FIG. 2 is a diagram of a present disclosure system embodiment.

FIG. 3 is a diagram of a present disclosure system embodiment.

FIG. 4 is a diagram of a present disclosure system embodiment.

FIG. 5 is a flow chart of present disclosure process steps that may be performed on an excised tissue specimen after excision but before pathological examination.

FIG. 6 is a flow chart of present disclosure process steps that may be performed on an excised tissue specimen after receipt of the tissue specimen in a pathology lab.

### DETAILED DISCLOSURE

The present disclosure is directed to a system and method for producing an image-based registration of an excised surgical specimen. The image-based registration provides a means for a pathology lab to verify that the physical orientation of an excised specimen within the pathology lab coincides with the physical orientation of that same specimen assigned by the surgeon (or other technician) upon removal of the specimen. The present disclosure provides numerous advantages over current practices; e.g., mitigation or elimination of specimen orientation error, enhanced/facilitated communications between the surgeon removing the specimen and the pathologist analyzing the specimen, eliminating chain of custody issues, enhanced record keeping, and others.

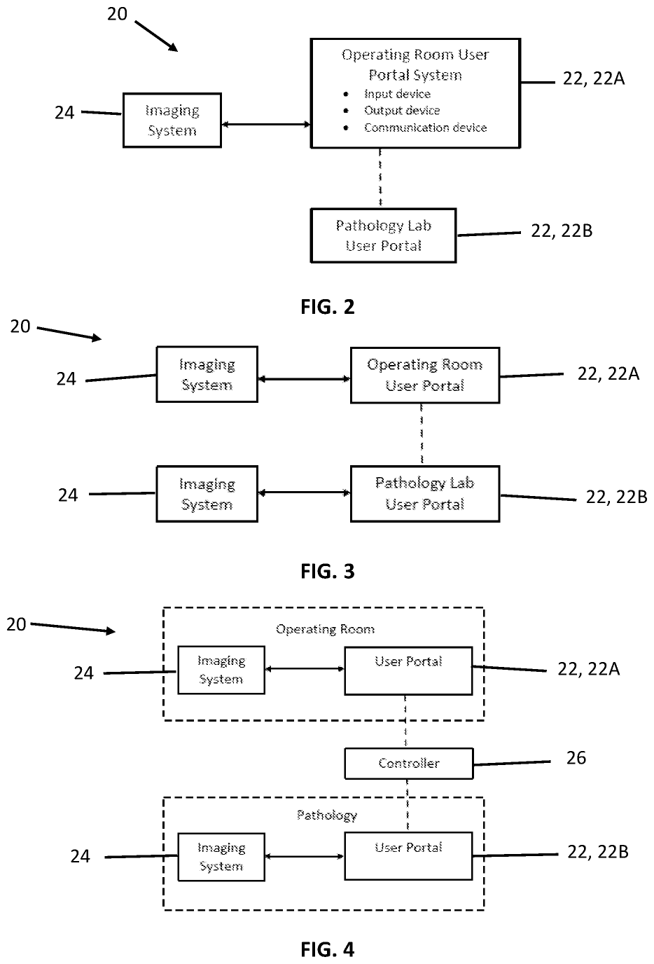

Referring to FIGS. 2-4, embodiments of the present disclosure system 20 may assume a variety of different configurations. For example, some configurations include one or more user portals 22, an imaging system 24, and a system controller 26. FIG. 2 diagrammatically illustrates a system 20 embodiment having a user portal 22 and an imaging system 24 located in or near an operating room. FIG. 3 diagrammatically illustrates a system 20 embodiment having a user portal 22 and an imaging system 24 located in or near an operating room and a user portal 22 and an imaging system 24 located in a pathology lab. FIG. 4 diagrammatically illustrates a system 20 embodiment having a user portal 22 and an imaging system 24 located in or near an operating room, a user portal 22 and an imaging system 24 located in a pathology lab, and a controller remotely located. These system embodiments are non-limiting examples of present disclosure systems 20.

The imaging system 24 is configured to produce three-dimensional digital images of a specimen resected from a subject. In some embodiments, the imaging system 24 may be configured to produce a three-dimensional image of the specimen in a single imaging event. For example, the imaging system 24 may utilize a stereo camera capable of producing three-dimensional digital image of a specimen in a single imaging event. As another example, the imaging system 24 may be configured to produce a three-dimensional image of the specimen from image scanning (e.g., a video) of the respective surfaces of the specimen. As another example, the imaging system 24 may be configured to produce a three-dimensional image of the specimen from a plurality of digital images taken from different perspectives that may be combined via software to produce the three-dimensional image. The individual digital images taken from different perspectives may be captured using a digital camera. The present disclosure is not limited to producing a digital three-dimensional image with any particular device or process; e.g., in addition to the embodiments described above, further embodiments may utilize a LIDAR camera or imaging technology that utilizes time-of-flight technology. In those imaging system 24 embodiments that utilize software to produce a three-dimensional image from a scanned image or a plurality of perspective images, the software used to produce the three-dimensional image from the imaging system 24 input may reside on a device separate from the imaging system 24; e.g., on a user portal 22.

In those embodiments of the present disclosure that include a user portal 22, the user portal 22 may include a controller, an input device, an output device, and an electronic communication device. A user portal 22 may be configured for use at a location where it can be used with the imaging system 24 to produce the aforesaid three-dimensional images of an excised tissue specimen (e.g., at or adjacent an operating room; e.g., see FIG. 2). The imaging system 24 may be in direct communication with the user portal 22 (e.g., either hardwired to or in wireless communication with the user portal 22) to permit direct transfer of image files there between. The input device (e.g., a GUI interface, a keyboard, voice command device, etc.) may be configured to permit a user to enter data and/or instructions and the like to accompany the produced three-dimensional image. For example, a technician may use the input device to modify image files (e.g., add labeling, annotation, etc.), append data (e.g., a surgical record, etc.) to the images, or add other data that may be subsequently used during pathological examination of the specimen. The output device may, for example, be configured to display information (e.g., a visual display of the image files and/or other related data) useful to the technician. The electronic communication device may be configured to electronically transfer data files by hardwire (e.g., via Ethernet connection) or by wireless connection (e.g., Bluetooth). In some embodiments, a personal computer (e.g., a laptop) may be used as a user portal 22.

In those embodiments wherein the present disclosure includes more than one user portal 22 (e.g., a first user portal 22A and a second user portal 22B), one or more second user portals 22B may be disposed at a location where it can be used during the pathological preparation and/or examination of the excised tissue specimen; e.g., at a grossing lab, or a pathology lab, etc. (e.g., see FIGS. 2-4) In these embodiments, the second user portal 22B may be configured similar to the user portal 22 described above.

In some embodiments, the system 20 may include a system controller 26; e.g., see FIG. 4. In these embodiments, the user portals 22 may be in communication with the system controller 26. In this manner, three-dimensional images produced using imaging system 24 may be communicated to a first user portal (e.g., at the operating room) and subsequently to the system controller 26. The three-dimensional images may be communicated to a second user portal 22B (e.g., at the pathology lab) through the system controller 26, or may be stored at the system controller 26 and accessed by the second portal from the system controller 26.

In some embodiments, the imaging system 24 may be in communication with the system controller 26 and configured to upload the image data to the system controller 26. In any of the present disclosure system 20 embodiments, the system controller 26 may be locally based or remotely based; e.g., cloud based.

The system controller 26 and the user portal controllers may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system 20 (or component) to accomplish the same algorithmically and/or coordination of system components. The respective controllers may include or be in communication with one or more memory devices. The present disclosure is not limited to any particular type of memory device, and the memory device may store instructions and/or data in a non-transitory manner. Examples of memory devices that may be used include read-only memory, random access memory, volatile memory, non-volatile memory, static memory, dynamic memory, flash memory, cache memory, and/or any device that stores digital information. Communications between the system components (including the system controller 26) may be via a hardwire connection or via a wireless connection.

### Operation:

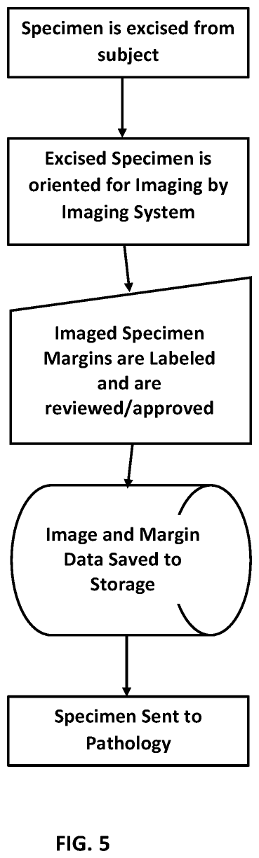

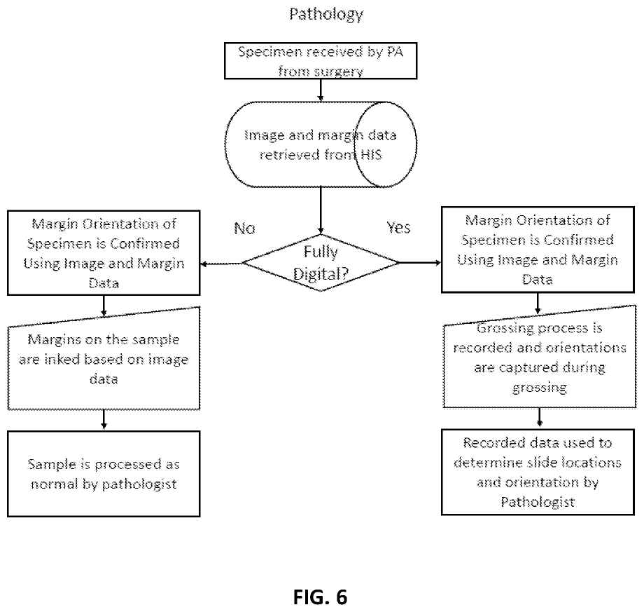

Referring to FIGS. 5 and 6, after a tissue specimen is excised, a technician at that location (e.g., in or near the operating room) may use the imaging system 24 to image the tissue specimen to produce the three dimensional image of the excised specimen. The manner in which the excised tissue is imaged may depend on the type of imaging system 24 used; e.g., a three-dimensional specimen image may be produced using a stereo camera, or a three-dimensional specimen image may be produced from a scan of the respective surfaces of the specimen, or a three-dimensional image may be produced using a plurality of digital images taken from different perspectives and software that combines the individual images into the three-dimensional image, or from a LIDAR camera, or from an imaging system that utilizes time-of-flight technology. As stated above, in some embodiments image files from the imaging system 24 may be communicated to a separate device (e.g., a user portal 22) where the three-dimensional image may be produced/displayed.

In some instances, prior to imaging the excised specimen, markers recognizable within an image (e.g., physical elements, fluorescent labels, quantum dots, fiducial markers, etc.) may be added to the excised specimen to facilitate identification of respective specimen surfaces and subsequent registration between a three-dimensional image of an excised specimen and the specimen itself.

In some instances, the technician (e.g., the surgeon or surgical assistant) may annotate the produced three-dimensional image with information useful in the pathology process. The annotation process may include adding virtual markers to the image of the excised specimen (which markers may be in place of or in addition to earlier applied markers), or labeling, or other information that may be useful for the record and/or during a pathology examination of the specimen. In those embodiments that include a user portal 22, a technician may perform the annotation/labeling using a user portal 22.

In some embodiments, the system 20 may be configured to permit the technician (e.g., the surgeon) to input data (e.g., a surgical record) that is communicated with the three-dimensional image. In this manner, the three-dimensional image and the surgical record may be correlated together and thereby prevent the two from being separated or mixed with other unrelated records. The electronic three-dimensional image file (and data file when included) facilitates the transfer of data between parties and enhances record keeping. In those embodiments that include a user portal 22, a technician may input the data that is communicated with the three-dimensional image using a user portal 22.

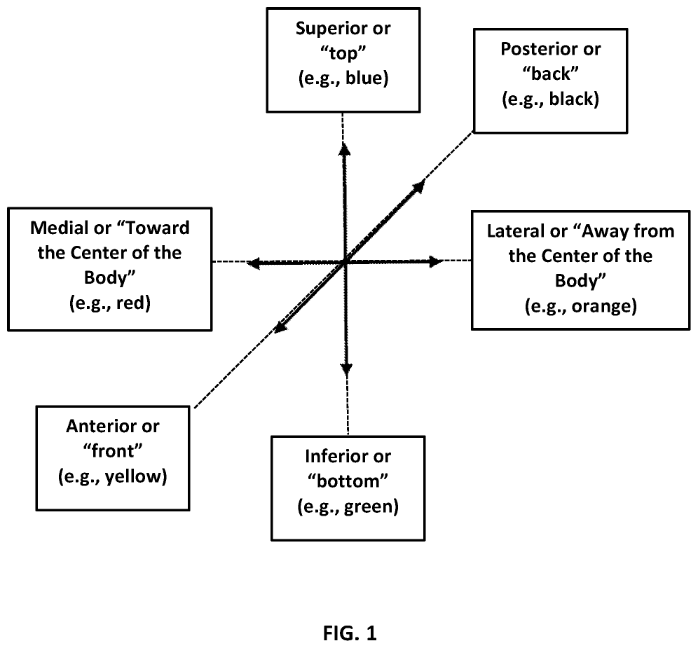

As indicated above and shown in FIG. 1, existing methods of indicating the orientation of an excised specimen may include applying different colorants to the respective surfaces of the excised specimen (e.g., medial, lateral, posterior, anterior, superior, and inferior surfaces). In some embodiments, the present disclosure system 20 may permit a technician (e.g., in the operating room or a pathology lab) to produce a copy of the three-dimensional image of the excised specimen with the respective colorants virtually applied to the respective surfaces. In this manner, a three dimensional image of the specimen may be provided in a manner consistent with current marking practices to give users a level of familiarity and comfort. In those embodiments that include a user portal 22, a technician may perform the virtual surface coloration using a user portal 22.

An advantage of the present disclosure is the flexibility and the enhanced “error-proofing” it provides. For example, an operating room technician other than the surgeon may operate the system 20 to produce the three-dimensional image, an identified orientation of the image (e.g., identifying surfaces as medial, lateral, posterior, anterior, superior, inferior, etc.), and related data (e.g., surgical record). The surgeon who excised the tissue specimen (or other technician) may subsequently review the orientation assigned to the three-dimensional image surfaces, the accompanying data, the virtual staining if used, and the like to ensure the information is complete and accurate.

The produced three-dimensional image may be sent to the pathology lab for analysis, or may be stored in a manner accessible by pathology technicians; e.g., stored in memory at the system controller 26 which may be cloud based. In those system 20 embodiments wherein one or more user portals 22 are located in a pathology lab, the three-dimensional image (and associated records) may be accessed at those user portals 22.

The excised tissue specimen is delivered to the pathology lab; e.g., the grossing lab section thereof. The present disclosure may be used to perform a registration between the three-dimensional images of the excised tissue specimen created subsequent to the specimen being excised (i.e., the “post-excised specimen three-dimensional image” created at the operating room) and the actual tissue specimen now arrived at the pathology lab (e.g., at the grossing lab) in a variety of different ways. For example, in some embodiments the system 20 may include a second imaging system 24 at the pathology lab configured to produce the same or a compatible three-dimensional image; e.g., a second three-dimensional image). In these embodiments, the initial three-dimensional image may be compared with the second three-dimensional image to verify the identity of the specimen and if verified, correlate the orientation of the tissue specimen surfaces with the tissue specimen surface orientation earlier defined. As another example where a second imaging system 24 is used, the tissue specimen may be viewed using a second imaging system 24 and the earlier produced three-dimensional image may be overlayed onto the tissue specimen then being viewed to confirm or adjust orientation, as necessary. A pathology technician can confirm whether the orientation of the tissue specimen (now located in pathology) agrees with the earlier assigned orientation of the tissue specimen surfaces (i.e., medial, lateral, posterior, anterior, superior, inferior, etc.) by aligning the view of the tissue specimen with the earlier produced three-dimensional image. The post-excised specimen three-dimensional image may assume a variety of different formats during the registration process. For example, in some embodiments, the post-excised specimen three-dimensional image may have a format that allows for an augmented reality (“AR”) registration approach. As another example, the post-excised specimen three-dimensional image may have a wireframe format that can be overlayed for registration purposes. As another example, markers (virtual or actual) captured in the post-excised specimen three-dimensional image may be used for registration purposes. The above examples of how registration may be accomplished between the post-excised specimen three-dimensional image and the tissue specimen as it resides in pathology are not intended to be all inclusive, and the present disclosure contemplates that alternative techniques may be used.

In some embodiments of the present disclosure, a pathology technician may identify the orientation of the tissue specimen (now located in pathology) and the respective tissue specimen surfaces (i.e., medial, lateral, posterior, anterior, superior, inferior, etc.) by visual comparison with the post-excised specimen three-dimensional image; e.g., visual comparison between the post-excised specimen three-dimensional image as displayed on a user portal 22 and the actual tissue specimen. If the excised tissue specimen has a distinctive geometry, the aforesaid visual comparison may be acceptable. The ability of the present disclosure system 20 embodiments to overlay the post-excised specimen three-dimensional image onto the actual tissue specimen residing in pathology, however, can provide a greater degree of certainty and consequent reduction in potential error.

Once the orientation of the excised tissue specimen is confirmed, the grossing lab technician may apply colorants to the specimen surfaces (i.e., “margins”) to facilitate further processing.

Some embodiments of the present disclosure may be configured to further facilitate the pathology examination of the excised tissue specimen. For example, in some instances a histopathological examination of a tissue specimen may include a procedure commonly referred to as “bread-loafing” that involves cutting the tissue specimen into three or more sections. The cut sections are mounted by embedding in paraffin or a frozen medium. The cut edge of the specimen is then typically thinly sliced using a microtome. The thin slices of the tissue specimen are then mounted on a glass slide, stained, and covered with another layer of glass. In embodiments of the present disclosure that include an imaging system 24 disposed to produce three-dimensional images of the tissue specimen during pathology, the imaging system 24 can be used to produce a three-dimensional image of the bread-loafed section and subsequently formed sections of the tissue specimen produced during the bread-loafing procedure. In some embodiments, the imaging system 24 can be used to capture and record images of tissue specimen slices as they process through pathology. For example, an imaging system 24 that is configured to produce a three-dimensional video of the tissue specimen during the pathology process can allow any portion of the tissue specimen to be later assessed by analyzing stop frames of the three-dimensional video from the recorded file. In these system 20 embodiments, the three-dimensional image data collected during the pathology process (e.g., during microtoming) can provide an additional means to verify the orientation of the tissue specimen sections is appropriate, and thereby mitigate or eliminate potential errors that may be associated with the orientation of the tissue specimen.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements.

1. A method of producing an image-based registration of an excised tissue specimen for histopathological examination, the method comprising:
producing a three-dimensional image of an excised tissue specimen having a plurality of surfaces at a first location, the excised tissue specimen having an in-vivo orientation prior to being excised;
producing orientation information relating to the orientation of the plurality of surfaces of the excised tissue specimen relative to the in-vivo orientation of the tissue specimen prior to being excised;
wherein the excised tissue specimen is transported to a second location and is positioned; and
using the three-dimensional image and the orientation information to confirm the excised tissue specimen positioned at the second location is in the in-vivo orientation.

2. The method of claim 1, wherein the second location is remote from the first location, and the method further comprises communicating the three-dimensional image and the orientation information to the second location for use in the confirmation step.

3. The method of claim 2, wherein the three-dimensional image is displayed on a user portal at the second location.

4. The method of claim 2, further comprising producing a second three-dimensional image of the excised tissue specimen at the second location, and using the second three-dimensional image in the confirmation step.

5. The method of claim 2, further comprising imaging the excised tissue specimen at the second location and overlaying the three-dimensional image of the excised tissue specimen onto the excised tissue specimen being imaged at the second location.

6. The method of claim 5, wherein the process of overlaying the three-dimensional image of the excised tissue specimen onto the excised tissue specimen being imaged at the second location is performed using augmented reality.

7. The method of claim 5, wherein the process of overlaying the three-dimensional image of the excised tissue specimen onto the excised tissue specimen being imaged at the second location includes alignment of a plurality of markers.

8. The method of claim 1, wherein the excised tissue specimen is sliced into a plurality of sections at the second location, the method further comprising producing a three-dimensional image of each of the plurality of sliced sections.

9. The method of claim 1, wherein the step of producing a three-dimensional image of an excised tissue specimen utilizes a stereo digital camera.

10. The method of claim 1, wherein the step of producing a three-dimensional image of an excised tissue specimen utilizes a scanning digital camera.

11. The method of claim 1, wherein the step of producing a three-dimensional image of an excised tissue specimen includes imaging the excised tissue specimen to produce a plurality of different perspective images and forming the three-dimensional image using the plurality of different perspective images.

12. The method of claim 1, further comprising virtually adding a plurality of different colorants to the plurality of surfaces within the three-dimensional image of an excised tissue specimen, wherein each said different colorant is added to a respective one of the plurality of surfaces.

13. A system for image-based registration of an excised tissue specimen for histopathological examination, the excised tissue specimen having a plurality of surfaces and an in-vivo orientation prior to being excised, the system comprising:
an imaging system configured to selectively produce a three-dimensional image of an excised tissue specimen;
a first user portal located in a first location; and
a second user portal located in a second location remote from the first location;
wherein the first user portal is in communication with the imaging system and the second user portal; and
wherein the first user portal includes an input device configured to permit entry of orientation information relating to the orientation of the plurality of surfaces of the excised tissue specimen, and the first user portal is configured to communicate the three-dimensional image and the orientation information to the second user portal;
wherein the second user portal is configured to permit a user to use the three-dimensional image and the orientation information to confirm the excised tissue specimen is in the in-vivo orientation.

14. The system of claim 13, wherein the second user portal includes a display configured to selectively display the three-dimensional image.

15. The system of claim 14, wherein the system further comprises a second imaging system at the second location, the second imaging system in communication with the second user portal, wherein the second imaging system is configured to produce a second three-dimensional image of the excised tissue specimen in a format that permits comparison between the first three-dimensional image and the second three-dimensional image.

16. The system of claim 14, wherein the system further comprises a second imaging system at the second location, the second imaging system in communication with the second user portal, wherein the second imaging system is configured to image the excised tissue specimen and overlay the three-dimensional image onto the imaged excised tissue specimen.

17. The system of claim 14, wherein the system further comprises a second imaging system at the second location, the second imaging system in communication with the second user portal, wherein the second imaging system is configured to overlay the three-dimensional image onto the imaged excised tissue specimen using augmented reality.

18. The system of claim 13, wherein the imaging system includes a stereo digital camera.

19. The system of claim 13, wherein the imaging system includes a scanning digital camera.

20. The system of claim 13, wherein the imaging system includes a digital camera and the second user portal is configured to form the three-dimensional image using images from the digital camera.
